(19) United States
(12) Patent Application Publication
(10) Pub. No.: US 2019/0125314 A1
(43) Pub. Date: May 2, 2019

(54) LIQUID SAMPLE CAPTURE APPARATUS

(71) Applicants: David R. Hall, Provo, UT (US); Joshua Larsen, Spanish Fork, UT (US); Jared Reynolds, Pleasant Grove, UT (US)

(72) Inventors: David R. Hall, Provo, UT (US); Joshua Larsen, Spanish Fork, UT (US); Jared Reynolds, Pleasant Grove, UT (US)

(21) Appl. No.: 16/233,090

(22) Filed: Dec. 27, 2018

Related U.S. Application Data
(63) Continuation-in-part of application No. 15/383,187, filed on Dec. 19, 2016.

(51) Int. Cl. A61B 10/00; G01N 1/20; G01N 1/44; G01N 33/493

(52) U.S. Cl. CPC A61B 10/007; G01N 1/20; A61B 2562/0271; G01N 33/493; G01N 1/44

(57) ABSTRACT

The liquid sample capture apparatus captures a sample of a liquid and holds the sample for optical analysis. The apparatus may include a mount which is traversed by a sample conduit. The mount may have an elongated shape and two ferrules may be placed within the mount along the longitudinal axis of the mount. An end of each of the ferrules may extend into the sample conduit forming a narrowing of the sample conduit which holds the liquid and prevents it from escaping prior to analysis. The opposite ends of the ferrules may each be connected to an optical cable. The optical cables may be connected to an analytical device, for example, a spectrometer. The apparatus may be mounted within a toilet bowl where it may capture and analyze a urine sample.

### CROSS REFERENCE TO RELATED APPLICATIONS

This application is a continuation in part of copending U.S. patent application Ser. No. 15/383,187 filed on Dec. 19, 2016 which is hereby incorporated by reference in its entirety.

### BACKGROUND

### Field of the Invention

This disclosure relates to apparatuses and methods for sampling liquids for analysis of the liquids, including urine.

### Background of the Invention

Liquid handling for analysis of the liquid may require tedious use of multiple devices to collect a sample of the liquid and then load the sample into an analytical device. In some situations, the liquid may be hazardous to handle.

Additional problems exist when the liquid is a bodily fluid. For example, collecting a urine sample for analysis is often inconvenient. Urine collection often involves urinating into a receptacle, such as a cup, and may result in unsanitary urine spills and drips. Some types of urinalyses require a user to urinate on a test strip or a specific part of a device. Controlling the urine stream to contact only a test strip or part of a device is often difficult and results in urine splashing onto unwanted areas. A device and method of collecting a urine sample that is easy and without risk of urine spills or drips is needed.

In addition, many types of urinalysis assays are not adapted for use outside the clinic setting. Consequently, a device and method of performing complex analysis of urine samples in the home or elsewhere outside of the clinic setting is needed.

### BRIEF SUMMARY OF THE INVENTION

We disclose an apparatus which captures liquid samples for analysis. The apparatus includes a mount with a sample conduit which traverse the mount from a top side to a bottom side of the mount. The sample conduit may include a first channel which is in fluid communication with a second channel. Two ferrules may be disposed within the mount, one on each side of the sample conduit. Each of the two ferrules may include a first end which extends into the sample conduit defining the second channel. The width of the second channel may be less than the width of the first channel. The varying widths between the first and second channels serve to prevent liquid from escaping the sample conduit after entering but prior to analysis.

The mount may have an elongated shape with a longitudinal axis. In some embodiments, the top side of the mount may slope upward forming a mound-shaped elevation which is elongated along the longitudinal axis. In some embodiments, the apex of the mound-shaped elevation is rounded while in others it is flattened forming substantially a triangular prism. In other embodiments, the top side of the mount slopes downward forming a dip into which liquid may flow.

The ferrules may be positioned parallel to the longitudinal axis. An optical cable may be inserted into a second end of each of the ferrules. These optical cables may be in connection with an analytical device, for example, a spectrometer, which may perform optical measurements of the liquid sample. The optical cables may include a single optical fiber or a plurality of optical fibers each of which may carry a different wavelength of light.

In each of the embodiments of the mount, the sample conduit may include a vertical slit. The vertical slit may run perpendicular to the longitudinal axis and through an apex of the mound-shaped elevation. The vertical slit may also run through the lowest point in a mount which includes a top side that slopes downward. A narrow slit may be useful to create a narrow path for light to pass through the sample during spectroscopic analysis.

Some embodiments include a temperature sensor which may be in thermal communication with the sample conduit. The temperature sensor may detect the temperature within the sample conduit.

Some embodiments include a heating device which may be in thermal communication with the sample conduit and with an electronics board. When the temperature sensor collects a temperature reading within the sample conduit which is below that which is desired for optimal optical readings, the electronics board may signal the heating device to actuate and heat the liquid in the sample conduit.

In some embodiments, the liquid sample capture apparatus includes a liquid-conveyance shell disposed adjacent to the top side of the mount. Liquid may travel along the liquid-conveyance shell toward the disclosed apparatus and direct the liquid toward the sample conduit. In an example, the liquid-conveyance shell is a wall of a toilet bowl. A user may urinate into the toilet bowl and the urine may flow down the wall of the toilet bowl towards the device.

The liquid-conveyance shell may include a third channel which traverses a thickness of the liquid-conveyance shell. The third channel may be in fluid communication with the sample conduit forming an extended sample conduit. The width of the third channel may be equal to or greater than the width of the first channel.

The apparatus may include a mounting mechanism, for example one or more bolts, which may attach the apparatus to a surface which is in contact with a liquid to be collected and analyzed. In an example, the apparatus may be attached to a wall of a toilet bowl.

Some embodiments may include an evacuation inlet which may be in fluid communication with the sample conduit. The evacuation inlet may be disposed within a fourth channel which is in fluid communication with the sample conduit. Gaseous or liquid materials may be injected into the sample conduit through the evacuation inlet to cleanse or dry the sample conduit between uses.

### BRIEF DESCRIPTION OF THE DRAWINGS

FIG. 1 illustrates a top-down perspective view of an embodiment of the disclosed liquid sample capture apparatus according to an embodiment of the disclosure.

FIG. 2 illustrates a top-down perspective view of an embodiment of the disclosed liquid sample capture apparatus including an attachment mechanism according to an embodiment of the disclosure.

FIG. 3 illustrates a cross-sectional view of an embodiment of the disclosed liquid sample capture apparatus according to an embodiment of the disclosure.

FIG. 4A illustrates a cross-sectional view of an embodiment of the disclosed liquid sample capture apparatus according to an embodiment of the disclosure.

FIG. 4B illustrates a close-up view of a region shown in FIG. 4A.

FIG. 5A illustrates a cross-sectional view of an embodiment of the disclosed liquid sample capture apparatus within a toilet bowl.

FIG. 5B illustrates a close-up view of a region shown in FIG. 5B.

FIG. 6 illustrates a close-up, top-down, perspective view of an embodiment of the sample conduit as part of an embodiment of the disclosed liquid sample capture apparatus.

FIG. 7 illustrates an alternative shape of the elevated portion of the base which may be included in an embodiment of the disclosure.

### DETAILED DESCRIPTION OF THE INVENTION

Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

We disclose an apparatus for capturing liquid samples for analysis. The apparatus may include a mount with a top side and a bottom side. The mount may include a sample conduit which traverses the mount from the top side to the bottom side of the mount. In some embodiments, the sample conduit is elongated and comprises a vertical slit. The mount may be an elongated structure which is longest at its longitudinal axis and the vertical slit may run perpendicular to the longitudinal axis. In some embodiments, the inner walls of the slit may be coated with a hydrophobic material which may make cleaning the slit between uses easier.

In some embodiments, a compliant, fluid-tight seal surrounds an opening leading into the sample conduit. This seal may inhibit liquid from being diverted from entering the channel and, instead, encourage the liquid to pool along the top side of the mount. As will be described in more detail, the sample conduit captures and holds a liquid sample during analysis. Consequently, the seal assists the liquid sample in entering the sample conduit for analysis.

In some embodiments, the mount may be constructed all, or partly, from aluminum. In other embodiments, the mount may be steel, stainless steel, and other metals and alloys know in the art. In some embodiments, the mount may be constructed all, or partly, from poly(methyl methacrylate), polystyrene, polyvinylchloride, and other synthetic polymers and co-polymers known in the art.

The mount may include an elevated top side which may slope upward to form an elongated, mound-shaped elevation. In some embodiments, the mound-shaped elevation may be substantially flattened at the apex forming a triangular pyramid. The sample conduit may be in the form of a vertical slit which may bisect the apex of a mound-shaped elevation which is substantially rounded at the apex or it may bisect the mound-shaped elevation at a point where it is substantially flattened.

In another embodiment, the top side of the mount may slope downward forming a dip into which liquid may flow. The sample conduit may be at the lowest region of the dip. In this embodiment, the mount may be elongated, and the sample conduit may bisect the mount at a plane which is perpendicular to the longitudinal axis. Liquid may flow along the downward slope and into the sample conduit.

Two ferrules may be inserted into the mount. In embodiments in which the mount is elongated, the ferrules may be positioned parallel to the longitudinal axis of the mount. In this embodiment, each ferrule may include a first end which extends into the sample conduit. The section of the sample conduit that is above the first ends of the ferrules, hereinafter, the “first channel,” may be defined by the mount. This section of the sample conduit that is between the ends of the ferrules, hereinafter, the “second channel,” may have a width which is narrower than that of the first conduit. This narrowing of the sample conduit may inhibit captured liquid from subsequently exiting the sample conduit prior to analysis.

In some embodiments, each of the two ferrules is in communication with an optical cable. The optical cables may be inserted into a second end of each ferrule which is opposite the first end. The optical cables may be in connection with an analytical device which may include a spectrometer. The spectrometer may detect absorption or transmission of one or more of the following: ultraviolet light, infrared light, near infrared light, or visible light.

Optical readings are often temperature sensitive. For at least this reason, some embodiments of the apparatus may include a temperature sensor which is in thermal communication with the sample conduit. The temperature sensor may be in communication with an electronics board which may control the temperature sensor. Some embodiments may include a heating device which may be in thermal communication with the sample conduit. When the temperature sensor collects a temperature reading within the sample conduit which is below that which is desired for optimal optical readings, the electronics board may signal the heating device to actuate and heat the liquid in the sample conduit. In an example, the liquid may be urine and the desired temperature for optical reading may be normal body temperature (approximately 37° C.).

In some embodiments, the apparatus includes an attachment mechanism. In an example, the attachment mechanism may be one or more screws or bolts. In an example, the attachment mechanism may be four bolts.

The liquid sample capture apparatus may be attached to a liquid-conveyance shell through the attachment mechanism. The liquid sample capture apparatus may be mounted onto the liquid-conveyance shell such that the top of the mount is adjacent to the liquid-conveyance shell. A third channel may traverse a thickness of the liquid-conveyance shell. The third channel may be in fluid communication with the sample conduit, for example, by being aligned with the first channel. In doing so, an extended sample conduit may be formed. The width of the third channel may be greater than or equal to the width of the first channel.

In some embodiments, the liquid-conveyance shell comprises a wall of a toilet bowl. The liquid sample may be urine which a user may deposit into the toilet bowl. The urine may flow down the wall of the toilet bowl and a sample of the urine may flow into the disclosed liquid sample capture apparatus. The urine sample may be analyzed using optical techniques as disclosed herein.

In some embodiments, the liquid sample capture apparatus may include an evacuation inlet. The evacuation inlet may be disposed within a fourth channel which is an extension of the sample conduit. Accordingly, the fourth channel may be in fluid communication with the sample conduit. In some embodiments, the fourth channel may lead into the second channel. In some embodiments the width of the fourth channel may be greater than the width of the second channel.

Liquids, which may include water, cleaning fluid, or disinfectants such as isopropanol, may be injected into the sample conduit through the evacuation inlet. This action may be performed to clean the sample conduit between samples. In addition or alternatively, air or other gaseous materials may be injected into the sample conduit to dry the sample conduit between uses. This action may be performed to blow out any residual liquid, including a cleansing liquid or liquid sample, to prepare the sample conduit for a background optical measurement. In addition, drying the sample conduit before collecting a liquid sample may prevent bubbles from forming on the sides of the sample conduit which may negatively impact the optical measurements. Furthermore, one or more reagents may be injected into the sample conduit through the evacuation inlet. These reagents may participate in a chemical reaction which may be used to detect a specific substance or analyte in the liquid sample

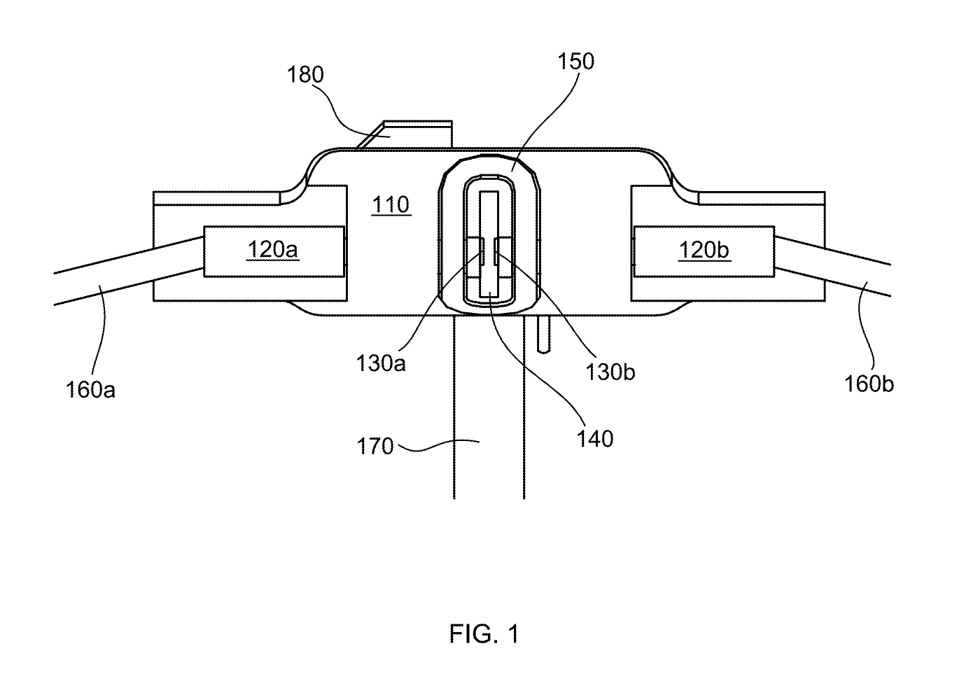

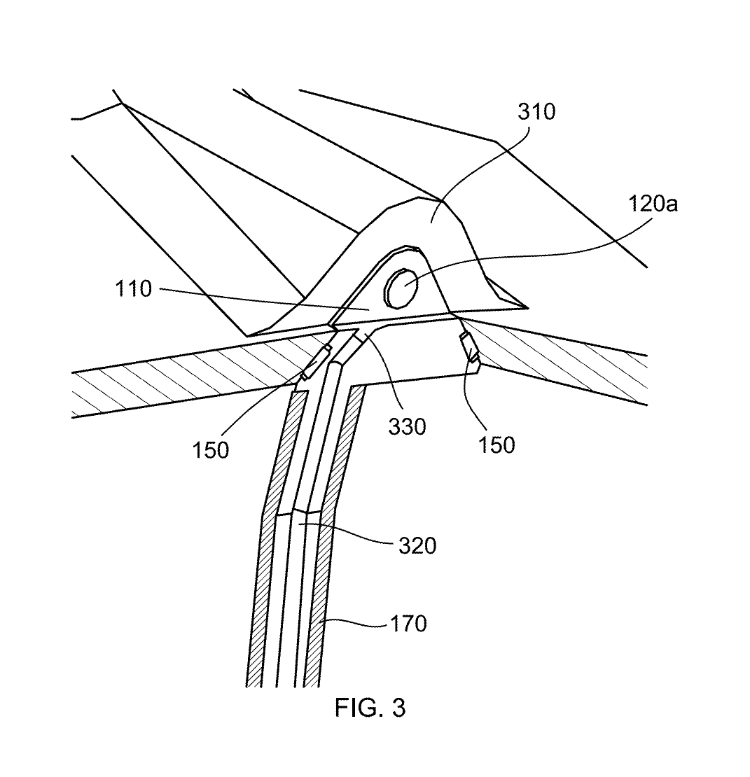

Referring now to the drawings, FIG. 1 illustrates an embodiment of the disclosed liquid capture apparatus as seen from an elevated position. The apparatus includes mount 110 into which ferrules 120a and 120b are inserted. Ferrule 120a includes ferrule first end 130a and ferrule 120b includes ferrule first end 130b. Each of ferrule first ends 130a and 130b extend into sample conduit 140. Ferrule first ends 130a and 130b create a narrowing in sample conduit 140, referred to herein as the second channel. Seal 150 surrounds the opening into sample conduit 140 keeping liquid from easily spilling out of the opening. Optical cables 160a and 160b are inserted into a second end of each of ferrules 130a and 130b respectively. Optical cables 160a and 160b may be part of a spectroscopic analytical device which may be in connection with the disclosed apparatus and which may analyze the liquid in sample conduit 140. Tube casing 170 houses a cleansing conduit (introduced as cleansing conduit 320 in FIG. 3) which extends upward into sample conduit 140.

The embodiment of FIG. 1 includes electronics board 180. Various embodiments discussed herein may include parts which require electronics to function. These electronics may be included in electronics board 180.

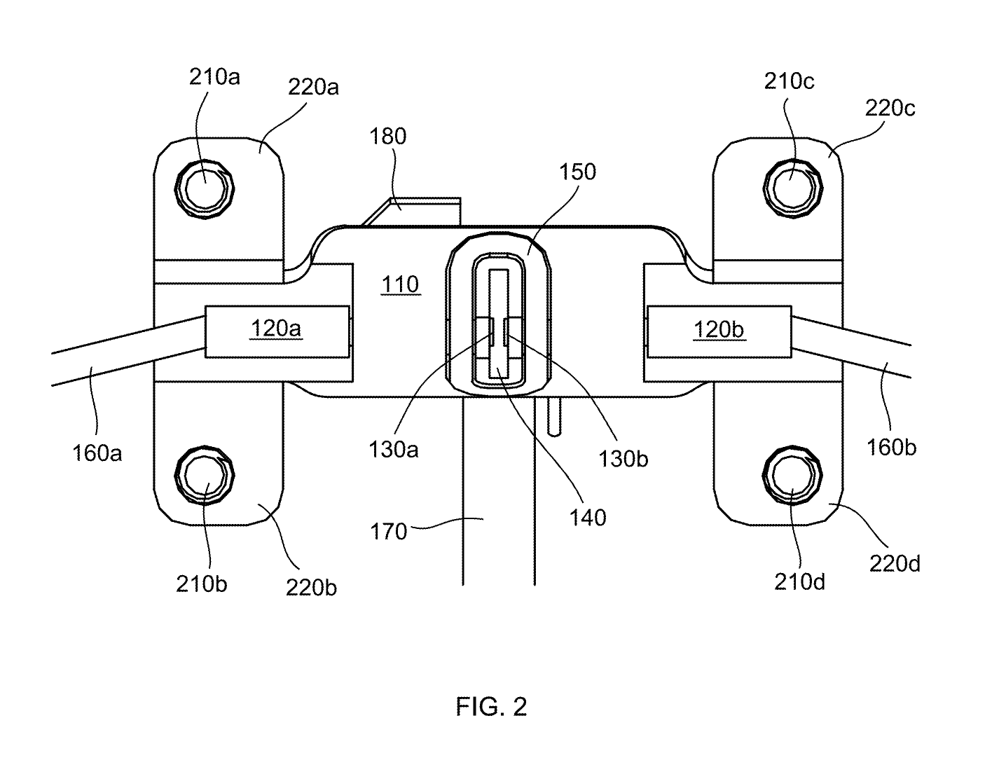

FIG. 2 is an embodiment similar to that shown in FIG. 1. FIG. 2 includes bolts 210a, 210b, 210c, and 210d which are inserted into flanges 220a, 220b, 220c, and 220d respectively. Flanges 220a, 220b, 220c, and 220d and bolts 210a, 210b, 210c, and 210d attach the apparatus to a surface which may be in contact with the liquid to be collected. In an example, the apparatus may be attached to a surface of a toilet bowl into which urine may be dispensed. The apparatus may collect and analyze the urine deposited into the toilet bowl.

FIG. 3 illustrates a cross sectional view embodiment of the disclosed apparatus installed beneath a toilet bowl wall where it is to collect urine. The cross-section through the apparatus as shown passes through sample conduit 140 and through ferrule 120a at its widest section, not including ferrule first end 130a.

Mount 110 is adjacent to toilet bowl wall 310. Note that toilet bowl wall 310 continues along the upward curve of the mound-shaped elevation in mount 110 forming a mound-shaped elevation in a region of toilet bowl wall 310. This mound-shaped elevation may act as a dam to detain urine so that the urine may enter sample conduit 140.

Tube casing 170 extends downward from sample conduit 140 as originally presented in FIG. 1. Tube channel 170 houses cleansing channel 320 which may transport water, electrolyzed water, air, isopropanol, or any other cleansing or rinsing material known in the art. Cleansing channel 320 is in fluid communication with evacuation inlet 330 which, in turn, is in fluid communication with sample conduit 140. Cleansing or rinsing material, which may be liquid or gaseous form, may be transported through cleansing channel 320 and enter sample conduit 140 through evacuation inlet 330.

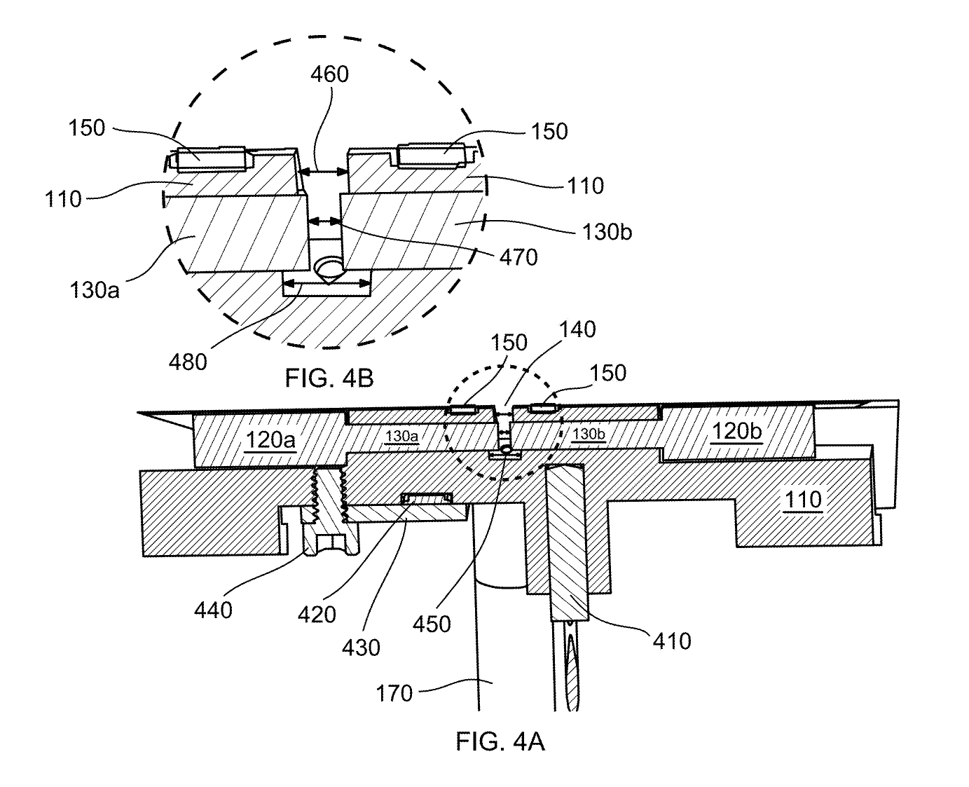

FIG. 4A illustrates a cross-sectional view of an embodiment of the apparatus. Unlike the embodiment of FIG. 3 in which mount 110 is shaped as a raised mound, the embodiment of FIG. 4A is substantially flat and does not include a mound-shaped elevation. Thus, instead of being blocked by the mound-shaped elevation to slow the liquid flow as it enters sample conduit 140, the liquid flows along a flat surface and into sample conduit 140 as the liquid passes over mount 110. Evacuation inlet 450 is an orifice through which air, water, or other cleansing or disinfecting liquid may be injected. Liquid or air may be injected through inlet 450 to rinse sample conduit 140 clean after use. In an example, isopropanol may be injected into evacuation inlet 450 to clean and disinfect sample conduit 140. Air may be injected into evacuation inlet 450 to blow out any residual liquid and to get a background measurement with the spectrometer with only air in sample conduit 140. Furthermore, drying sample conduit 140 prior to collecting a new sample may prevent bubbles from forming on the sides sample conduit 140 which may negatively impact spectrometer readings.

Additionally, the embodiment of FIG. 4A includes heater 410 which may heat the liquid sample to a desired temperature in order to acquire consistent spectroscopic readings. The embodiment of FIG. 4A further includes temperature sensor 420 which is in connection with electronics board 430. Temperature sensor 420 may detect the current temperature of the liquid that has entered through sample conduit 140.

Electronics board 430 is also in electrical communication with heater 410. If the temperature of the liquid is below the desired temperature, electronics board 430 actuates heater 410 to elevate the temperature of the liquid. In an example, the liquid may be urine and the desired temperature may be body temperature (approximately 37° C.). In this embodiment, electronics board 430 is secured by bolt 440.

FIG. 4B is a close-up view of the region within the dashed circle shown in FIG. 4A. First channel 460 is part of sample conduit 140 which is disposed between sections of mount 110. Below first channel 460 and within sample conduit 140 is second channel 470 which is disposed between ferrule first end 130a and ferrule first end 130b. Second channel 470 is narrower than first channel 460. Further down sample conduit 140 is shown third channel 480 which is disposed between sections of mount 110 and which is below second channel 470 in sample conduit 140. Third channel 480 is greater than or equal in width relative to first channel 460. This design improves liquid collection into sample conduit 140 and may prevent liquid from rebounding out of sample conduit 140 after collection.

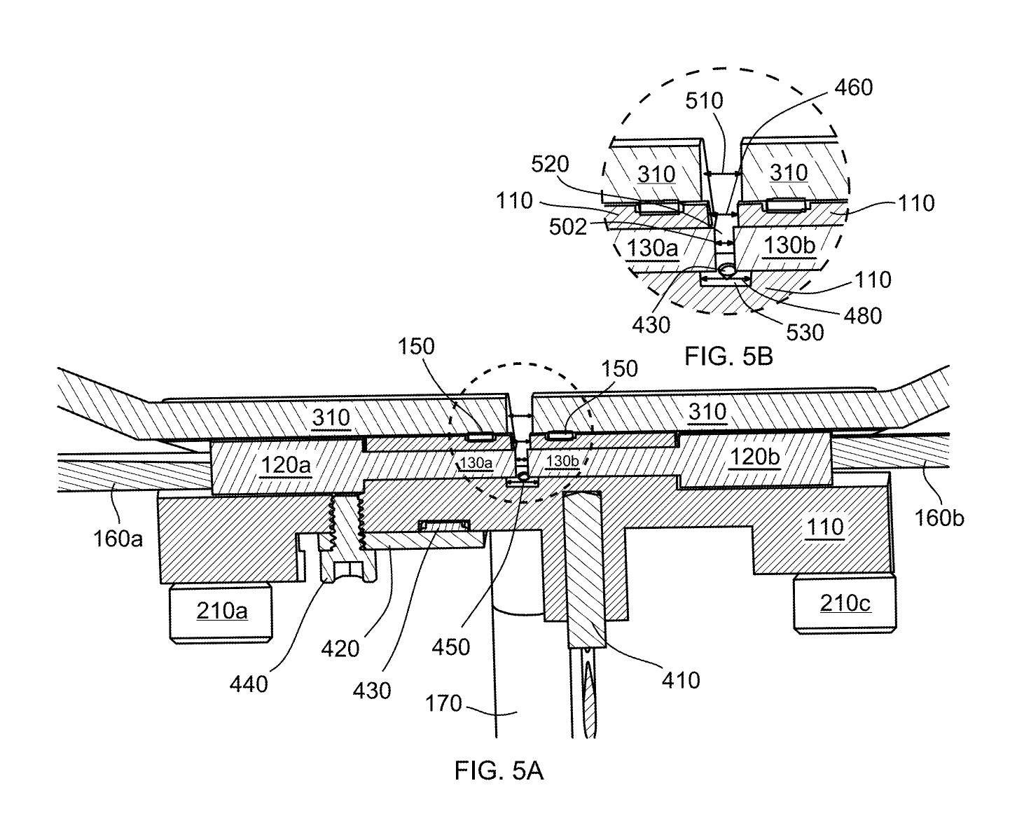

FIG. 5A illustrates a cross-sectional view of the embodiment of FIG. 4A which is installed beneath toilet bowl wall 310. Fourth channel 510 is part of sample conduit 140 and is defined by the surrounding sections of toilet bowl wall 310. Fourth channel 510 is approximately equal in width to first channel 460.

Urine may be deposited into a toilet that includes toilet bowl wall 310 and flow down toilet bowl wall 310 into fourth channel 510. The urine may then continue to pass through sample conduit 140 as illustrated in FIG. 4B. The most significant difference between the embodiment of FIGS. 4A/B and FIGS. 5A/B is the addition of toilet bowl wall 310 beneath which the embodiment of FIGS. 5A/5B is mounted and the addition of fourth channel 510 which lengthens sample conduit 140.

FIG. 5B is a close-up view of the region within the dashed circle shown in FIG. 5A. Similar to FIG. 4B, FIG. 5B provides a detailed illustration of different sections of sample conduit 140. Fourth channel 510 is shown as an orifice within toilet bowl wall 310. In this embodiment, fourth channel 510 is slightly wider than gap 460.

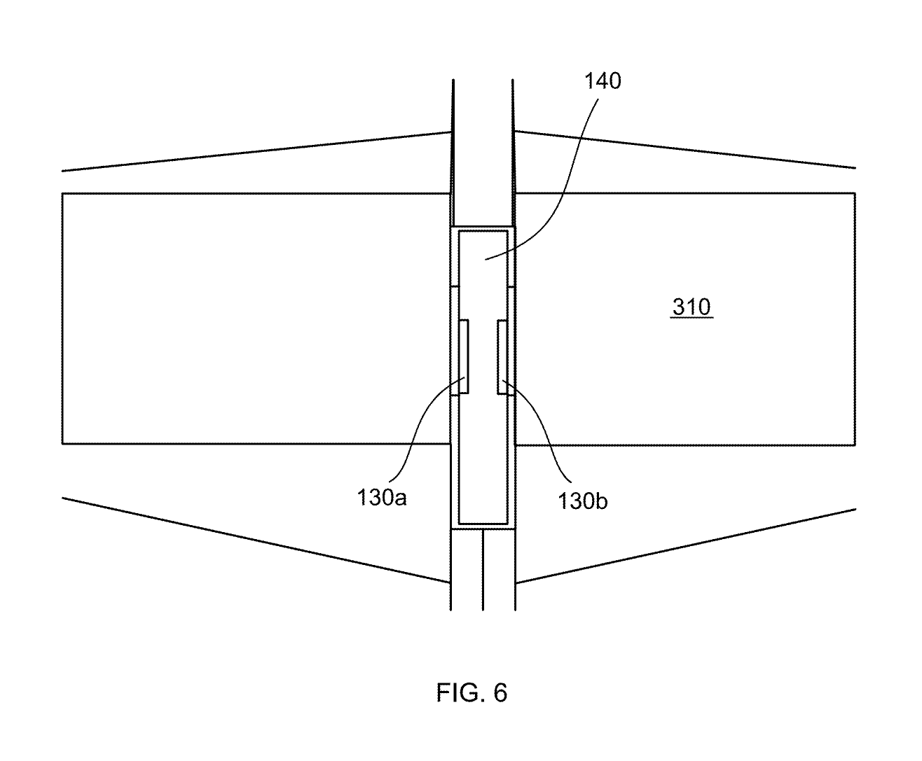

FIG. 6 is an elevated view of an embodiment of the apparatus. The device has been mounted within toilet bowl 310. Sample conduit 140 is shown with the narrowing caused by ferrule first ends 130a and 130b. Unlike previous embodiments, the apparatus of FIG. 6 is mounted at the bottom of a concave depression in toilet bowl wall 310. Mount 110 slopes downward forming a dip. Sample conduit 140 is at the lowest point in the dip. Urine may flow down the sloped region of toilet bowl wall 310 and into sample conduit 140.

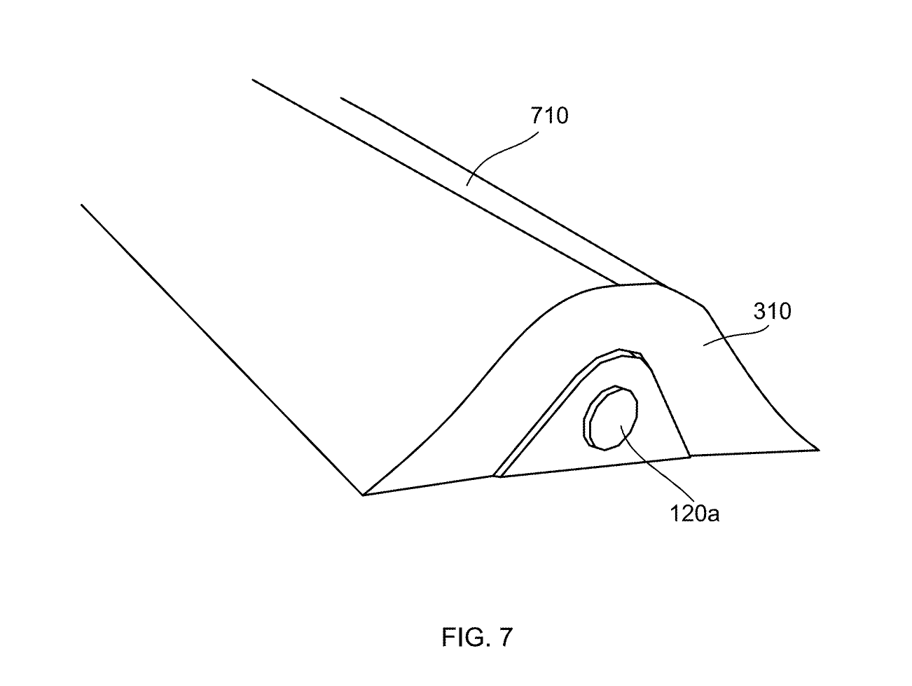

FIG. 7 illustrates a cross-sectional view of an embodiment of the disclosed apparatus in which mount 110 is shaped as a mound-shaped elevation. The peak of the mound-shaped elevation includes flattened area 710. This is in contrast to the more rounded peak of the mound-shaped elevation shown in FIG. 3. These two designs will have different liquid management capabilities and each may be combined with parts of other embodiments presented herein.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A liquid sample capture apparatus, comprising:
a. a mount, comprising:
i. a top side;
ii. a bottom side; and
b. a sample conduit, comprising a first channel and a second channel,
i. wherein the sample conduit bisects the mount extending from the top side to the bottom side of the mount,
ii. wherein the first channel is in fluid communication the second channel; and
c. two ferrules, wherein each of the two ferrules is disposed within the mount on either side of the sample conduit, wherein first end of each of the two ferrules extends into the sample conduit and defines the second channel, and wherein a width of the second channel is less than a width of the first channel.

2. The liquid sample capture apparatus of claim 1, further comprising a compliant, fluid-tight seal disposed on the top side of the mount and surrounding an opening into the first channel.

3. The liquid sample capture apparatus of claim 1, further comprising a first and a second optical cable, wherein the first and the second optical cables are each inserted into a second end of one of the two ferrules.

4. The liquid sample capture apparatus of claim 3, wherein the first and the second optical cables each comprise a plurality of optical fibers.

5. The liquid sample capture apparatus of claim 1, further comprising at least one attachment mechanism.

6. The liquid sample capture apparatus of claim 1, further comprising a liquid-conveyance shell, the liquid conveyance shell comprising a third channel,
a. wherein the liquid-conveyance shell is disposed adjacent to the top side of the mount,
b. wherein the third channel bisects the liquid-conveyance shell,
c. wherein the third channel is in fluid communication with the sample conduit forming an extended sample conduit, and
d. wherein a width of the third channel is greater than or equal to the width of the first channel.

7. The liquid sample capture apparatus of claim 6, wherein the liquid-conveyance shell comprises section of a wall of a toilet bowl.

8. The liquid sample capture apparatus of claim 1, wherein the mount comprises an elongated shape along a longitudinal axis, and wherein the longitudinal axis is substantially parallel to the two ferrules.

9. The liquid sample capture apparatus of claim 8, wherein the top side of the mount slopes upward forming a mound-shaped elevation, and wherein the mound-shaped elevation is elongated along the longitudinal axis.

10. The liquid sample capture apparatus of claim 8, wherein the sample conduit comprises a slit, and wherein the slit runs perpendicular to the longitudinal axis and bisects an apex of the mound-shaped elevation.

11. The liquid sample capture apparatus of claim 8, wherein the top side of the mount slopes upward relative to the bottom side of the mount, and wherein an apex of the mound-shaped elevation is substantially flattened, substantially forming a triangular prism.

12. The liquid sample capture apparatus of claim 11, wherein the sample conduit comprises a vertical slit, wherein the vertical slit runs perpendicular to the longitudinal axis of the mount and bisects the apex of the mound-shaped elevation.

13. The liquid sample capture apparatus of claim 1, wherein the top side of the mount comprises a downward dip toward the bottom side of the mount.

14. The liquid sample capture apparatus of claim 1, wherein the top side of the mount is substantially flat.

15. The liquid sample capture apparatus of claim 1, wherein the mount is constructed of a material comprising aluminum.

16. The liquid sample capture apparatus of claim 1, further comprising an evacuation inlet in fluid communication with the sample conduit.

17. The liquid sample capture apparatus of claim 16, wherein the evacuation inlet is disposed within a fourth channel, wherein the fourth channel is in fluid communication with the sample conduit.

18. The liquid sample capture apparatus of claim 17, wherein the fourth channel comprises a width which is greater than a width of the second channel.

19. The liquid sample capture apparatus of claim 1, further comprising a heating device in thermal communication with the sample conduit.

20. The liquid sample capture apparatus of claim 1, further comprising a temperature sensor, wherein the temperature sensor is in thermal communication with the sample conduit.
